(19) United States
(12) Patent Application Publication
(10) Pub. No.: US 2025/0217811 A1
(43) Pub. Date: Jul. 3, 2025

(54) METHODS, SYSTEMS, AND COMPUTER PROGRAM PRODUCTS FOR PREDICTING WHEN PRIOR AUTHORIZATION IS REQUIRED FOR A HEALTH CARE PROCEDURE USING STATISTICAL ANALYTICS

(71) Applicant: Optum, Inc., Minnetonka, MN (US)

(72) Inventors: Ting-Yu Ho, Shoreline, WA (US); Guohua M. Zhao, Princeton, NJ (US); Letitia Murr, Charlotte, NC (US); Feili Yu, Shoreline, WA (US); Michael F. Neale, Sebastian, FL (US); John D. Evans, Alpharetta, GA (US); Mark J. Fleming, Fitchburg, WI (US)

(21) Appl. No.: 18/400,184

(22) Filed: Dec. 29, 2023

(51) Int. Cl. G06Q 20/40

(52) U.S. Cl. CPC G06Q 20/405

(57) ABSTRACT

A method includes processing, by one or more processors, historical claim and claim remittance information to extract prior authorization data; performing, by the one or more processors, a probabilistic analysis on the prior authorization data for a procedure to generate a first metric and a second metric, the first metric comprising a percentage of claims including the procedure in which prior authorization is applied for and the second metric comprising a percentage of claims including the procedure in which prior authorization was not applied for and were denied payment for not applying for prior authorization; determining, by the one or more processors, whether the first metric satisfies a first authorization threshold and the second metric satisfies a second authorization threshold, the first and second thresholds being associated with a prior authorization rule corresponding to the procedure; and generating, by the one or more processors, a recommendation on whether to obtain prior authorization before performing the procedure based on whether the first metric satisfies the first authorization threshold and the second metric satisfies the second authorization threshold.

### FIELD

The present inventive concepts relate generally to health care systems and services and, more particularly, to predicting when prior authorization is required before performing a health care procedure.

### BACKGROUND

Health care service providers have patients that pay for their care using a variety of different payors. For example, a medical facility or practice may serve patients that pay by way of different insurance companies including, but not limited to, private insurance plans, government insurance plans, such as Medicare, Medicaid, and state or federal public employee insurance plans, and/or hybrid insurance plans, such as those that are sold through the Affordable Care Act. Some procedures performed by a provider, however, may require prior authorization or approval by a payor before the procedure is performed. Determining whether a specific procedure requires authorization, however, may be complex. Some providers may use a manual process to contact a payor to inquire whether a procedure requires prior authorization before it is performed. Providers may use the manual process because different payors may have different prior authorization policies and rules and it may not be simple or straightforward to obtain the prior authorization policies and rules from the various payor sites. For example, some payors may have complicated documents that describe their prior authorization policies and rules, some payors may provide an online lookup portal for searching for prior authorization policies and rules, while other payors may not provide any information on their prior authorization policies and rules.

### SUMMARY

According to some embodiments of the disclosure, a computer-implemented method comprises: processing, by one or more processors, historical claim and claim remittance information to extract prior authorization data; performing, by the one or more processors, a probabilistic analysis on the prior authorization data for a procedure to generate a first metric and a second metric, the first metric comprising a percentage of claims including the procedure in which prior authorization is applied for and the second metric comprising a percentage of claims including the procedure in which prior authorization was not applied for and were denied payment for not applying for prior authorization; determining, by the one or more processors, whether the first metric satisfies a first authorization threshold and the second metric satisfies a second authorization threshold, the first and second thresholds being associated with a prior authorization rule corresponding to the procedure; and generating, by the one or more processors, a recommendation on whether to obtain prior authorization before performing the procedure based on whether the first metric satisfies the first authorization threshold and the second metric satisfies the second authorization threshold.

In other embodiments, the method further comprises: electronically receiving, by the one or more processors, a prior authorization inquiry from a provider regarding the procedure; and electronically communicating, by the one or more processors, the recommendation to the provider in response to receiving the inquiry.

In still other embodiments, the recommendation comprises a recommendation to delay performing the procedure until the procedure is authorized by a payor.

In still other embodiments, the method further comprises: generating, by the one or more processors, a precision metric for the prior authorization rule; wherein the precision metric is given by a first quotient where a number of true positive events for the prior authorization rule is the dividend and a sum of true positive events plus false positive events for the prior authorization rule is the divisor when the prior authorization rule specifies that prior authorization is required; and wherein the precision metric is given by a second quotient where a number of true negative events for the prior authorization rule is the dividend and a sum of true negative events plus false positive events for the prior authorization rule is the divisor when the prior authorization rule specifies that prior authorization is not required.

In still other embodiments, the method further comprises: determining, by the one or more processors, the prior authorization rule based on the first metric and the second metric.

In still other embodiments, the method further comprises: electronically receiving, by the one or more processors, the prior authorization rule from a payor.

In still other embodiments, the method further comprises: determining, by the one or more processors, whether the precision metric satisfies a precision metric threshold; and generating, by the one or more processors, a recommendation for modifying the prior authorization rule when the precision metric satisfies the precision metric threshold.

In still other embodiments, the method further comprises: electronically modifying, by the one or more processors, the prior authorization rule responsive to generating the recommendation for modifying the prior authorization rule.

In still other embodiments, the method further comprises: electronically communicating, by the one or more processors, the recommendation for modifying the prior authorization rule to a payor; electronically receiving, by the one or more processors, a request from the payor to modify the prior authorization rule; and electronically modifying, by the one or more processors, the prior authorization rule responsive to receiving the request from the payor to modify the prior authorization rule.

In still other embodiments, the method further comprises: filtering, by the one or more processors, the historical claim and claim remittance information based on a plurality of features to remove a portion of the historical claim and claim remittance information leaving a subset of the historical claim and claim remittance information; wherein processing the historical claim and claim remittance information to extract the prior authorization data comprises: processing, by the one or more processors, the subset of the historical claim and claim remittance information to extract the prior authorization data.

In still other embodiments, the plurality of features comprise procedure code, payor name, payor identification, claim adjustment reason code, remittance advice remark code, principal diagnosis code, other diagnosis code, place of service, patient age, service year, service month, rendering provider taxonomy code, submitter identification, billing provider National Provider Identification (NPI), rendering provider NPI, or referring provider NPI.

In still other embodiments, the method further comprises: performing, by the one or more processors, a first statistical hypothesis test on the plurality of features to identify a first one of the plurality of features having a greatest impact on the first metric; and performing, by the one or more processors, a second statistical hypothesis test on the plurality of features to identify a second one of the plurality of features having a greatest impact on the second metric.

In still other embodiments, the statistical hypothesis test is a chi-squared test.

In still other embodiments, the probabilistic analysis is a Bayesian statistical analysis or an unsupervised machine learning analysis.

In some embodiments of the disclosure, a system comprises: one or more processors; and a memory coupled to the one or more processors and comprising computer readable program code embodied in the memory that is executable by the processor to perform operations comprising: processing, by the one or more processors, historical claim and claim remittance information to extract prior authorization data; performing, by the one or more processors, a probabilistic analysis on the prior authorization data for a procedure to generate a first metric and a second metric, the first metric comprising a percentage of claims including the procedure in which prior authorization is applied for and the second metric comprising a percentage of claims including the procedure in which prior authorization was not applied for and were denied payment for not applying for prior authorization; determining, by the one or more processors, whether the first metric satisfies a first authorization threshold and the second metric satisfies a second authorization threshold, the first and second thresholds being associated with a prior authorization rule corresponding to the procedure; and generating, by the one or more processors, a recommendation on whether to obtain prior authorization before performing the procedure based on whether the first metric satisfies the first authorization threshold and the second metric satisfies the second authorization threshold.

In further embodiments, the operations further comprise: generating, by the one or more processors, a precision metric for the prior authorization rule; wherein the precision metric is given by a first quotient where a number of true positive events for the prior authorization rule is the dividend and a sum of true positive events plus false positive events for the prior authorization rule is the divisor when the prior authorization rule specifies that prior authorization is required; and wherein the precision metric is given by a second quotient where a number of true negative events for the prior authorization rule is the dividend and a sum of true negative events plus false positive events for the prior authorization rule is the divisor when the prior authorization rule specifies that prior authorization is not required.

In still further embodiments, the operations further comprise: determining, by the one or more processors, whether the precision metric satisfies a precision metric threshold; and generating, by the one or more processors, a recommendation for modifying the prior authorization rule when the precision metric satisfies the precision metric threshold.

In still further embodiments, the operations further comprise: filtering, by the one or more processors, the historical claim and claim remittance information based on a plurality of features to remove a portion of the historical claim and claim remittance information leaving a subset of the historical claim and claim remittance information; wherein processing the historical claim and claim remittance information to extract the prior authorization data comprises: processing, by the one or more processors, the subset of the historical claim and claim remittance information to extract the prior authorization data.

In still further embodiments, the plurality of features comprise procedure code, payor name, payor identification, claim adjustment reason code, remittance advice remark code, principal diagnosis code, other diagnosis code, place of service, patient age, service year, service month, rendering provider taxonomy code, submitter identification, billing provider National Provider Identification (NPI), rendering provider NPI, or referring provider NPI; and the operations further comprise: performing, by the one or more processors, a first statistical hypothesis test on the plurality of features to identify a first one of the plurality of features having a greatest impact on the first metric; and performing, by the one or more processors, a second statistical hypothesis test on the plurality of features to identify a second one of the plurality of features having a greatest impact on the second metric.

In some embodiments of the disclosure, a computer program product comprises one or more non-transitory computer readable storage media including instructions that, when executed by one or more processors, cause the one or more processors to: process, by the one or more processors, historical claim and claim remittance information to extract prior authorization data; perform, by the one or more processors, a probabilistic analysis on the prior authorization data for a procedure to generate a first metric and a second metric, the first metric comprising a percentage of claims including the procedure in which prior authorization is applied for and the second metric comprising a percentage of claims including the procedure in which prior authorization was not applied for and were denied payment for not applying for prior authorization; determine, by the one or more processors, whether the first metric satisfies a first authorization threshold and the second metric satisfies a second authorization threshold, the first and second thresholds being associated with a prior authorization rule corresponding to the procedure; and generate, by the one or more processors, a recommendation on whether to obtain prior authorization before performing the procedure based on whether the first metric satisfies the first authorization threshold and the second metric satisfies the second authorization threshold.

Other methods, systems, articles of manufacture, and/or computer program products according to embodiments of the disclosure will be or become apparent to one with skill in the art upon review of the following drawings and detailed description. It is intended that all such additional systems, methods, articles of manufacture, and/or computer program products be included within this description, be within the scope of the present inventive subject matter and be protected by the accompanying claims.

### BRIEF DESCRIPTION OF THE DRAWINGS

Other features of embodiments will be more readily understood from the following detailed description of specific embodiments thereof when read in conjunction with the accompanying drawings, in which:

FIG. 1 is a block diagram that illustrates a communication network including a prior authorization prediction system in accordance with some embodiments of the disclosure;

FIG. 2 is a block diagram that illustrates the prior authorization prediction system in accordance with some embodiments of the disclosure;

FIG. 3 is a flowchart that illustrates operations prior authorization prediction system in accordance with some embodiments of the disclosure;

FIG. 4 is a chart with examples of Claim Adjustment Reason Codes (CARCs) in accordance with some embodiments of the disclosure;

FIG. 5 is a graph showing first and second metrics used in generating a prior authorization recommendation in accordance with some embodiments of the disclosure;

FIG. 6 is a table illustrating calculation of the first and second metrics along with a precision metric used in generating a prior authorization prediction for a rule in which prior authorization is required in accordance with some embodiments of the disclosure;

FIG. 7 is a table illustrating calculation of the first and second metrics along with a precision metric used in generating a prior authorization prediction for a rule in which prior authorization is not required in accordance with some embodiments of the disclosure;

FIG. 8 is a table illustrating calculation of precision metrics for multiple prior authorization rules associated with a particular health care area in accordance with some embodiments of the disclosure;

FIG. 9 is a table illustrating identification of features having greatest effect on the first and second metrics used in generating a prior authorization prediction in accordance with some embodiments of the disclosure;

FIG. 10 is a data processing system that may be used to prior authorization prediction system in accordance with some embodiments of the disclosure; and

FIG. 11 is a block diagram that illustrates a software/hardware architecture for use in in a prior authorization prediction system in accordance with some embodiments of the disclosure.

### DETAILED DESCRIPTION

In the following detailed description, numerous specific details are set forth to provide a thorough understanding of embodiments of the disclosure. However, it will be understood by those skilled in the art that embodiments of the disclosure may be practiced without these specific details. In some instances, well-known methods, procedures, components, and circuits have not been described in detail so as not to obscure the inventive concept. It is intended that all embodiments disclosed herein can be implemented separately or combined in any way and/or combination. Aspects described with respect to one embodiment may be incorporated in different embodiments although not specifically described relative thereto. That is, all embodiments and/or features of any embodiments can be combined in any way and/or combination.

As used herein, the term “provider” may mean any person or entity involved in providing health care products and/or services to a patient.

As used herein, a “service” includes, but is not limited to, a software and/or hardware service, such as cloud services in which software, platforms, and infrastructure are provided remotely through, for example, the Internet. A service may be provided using Software as a Service (SaaS), Platform as a Service (PaaS), and/or Infrastructure as a Service (IaaS) delivery models. In the SaaS model, customers generally access software residing in the cloud using a thin client, such as a browser, for example. In the PaaS model, the customer typically creates and deploys the software in the cloud sometimes using tools, libraries, and routines provided through the cloud service provider. The cloud service provider may provide the network, servers, storage, and other tools used to host the customer's application(s). In the IaaS model, the cloud service provider provides physical and/or virtual machines along with hypervisor(s). The customer installs operating system images along with application software on the physical and/or virtual infrastructure provided by the cloud service provider.

As used herein a “procedure” may be, but is not limited to, any type of treatment provided by a provider to a patient or any type of medicine or product prescribed or given to a patient for treatment. In general, a “procedure” may be defined as any activity directed at or performed on an individual with the object of improving health, treating disease or injury, or making a diagnosis.

Some embodiments of the disclosure stem from a realization that the use of an intermediary located in the cloud, such as a clearinghouse for processing claims generated by providers, may be configured to collect historical claim and claim remittance information for various payors, providers, and procedures, perform a probabilistic analysis, such as a Bayesian statistical analysis or an unsupervised machine learning analysis, on this historical information, and generate a prior authorization model that can be used to predict whether a particular procedure will require prior authorization based on a specific payor's prior authorization rules. Advantageously, this may alleviate the provider from the from the manual and potentially time consuming and expensive burden of contacting a payor to inquire whether a procedure requires prior authorization. Moreover, the support personnel at a payor may not always be familiar with the intricacies of their own prior authorization policies and rules. As a result, a provider may receive inaccurate information despite contacting a payor directly to confirm whether prior authorization is required for a procedure. Advantageously, the statistical analysis on historical claim and remittance information performed for a particular payor may provide a more accurate interpretation of a prior authorization policy or rule. As a result, the number of rejected claims may be reduced thereby reducing the processing load on the claim generation system at the provider and the claim processing system at the payor. In some embodiments, the prior authorization prediction service may generate a precision metric, which is an indication of how consistent a particular prior authorization rule is being enforced by a payor. If a prior authorization rule for a particular procedure has a precision metric that satisfies a particular precision metric threshold, then a recommendation may be made to modify the prior authorization rule so it may be enforced in a more consistent manner. Advantageously, this may again reduce the number of rejected claims thereby reducing the processing load on the claim generation system at the provider and the claim processing system at the payor. In some embodiments, a statistical hypothesis test may be used to determine which features associated with the claim, provider, and/or the patient have the most impact in the two metrics used to predict whether a procedure will require prior authorization. By identifying these features for rules in which prior authorization is required or prior authorization is not required, procedures in which these features are implicated may be given heightened scrutiny by providers and/or payors in considering whether prior authorization is required or not required. Although embodiments are described herein with respect to generating a prediction for whether a health care provider needs prior authorization before performing a procedure based on one or rules associated with a payor and payor plan, the embodiments described herein can be applied to generally to any environment in which authorization may be required before performing an act. For example, a health care provider may need authorization before performing a procedure from an authoritative entity, such as a medical review committee, or other entity, such as patient representative. Similar data may be obtained based on prior approvals or denials to generate metrics used for predicting whether a procedure requires prior authorization. Advantageously, embodiments of the disclosure may be applied generally to control the timing of operations that may be dependent on prior authorization before they are permitted to be performed. By generating predictions on whether prior authorization is needed, both manual and computing resources may be conserved in avoiding the processing of unnecessary prior authorization requests and in processing payment denials, penalties, and the like when operations are performed before authorization is received.

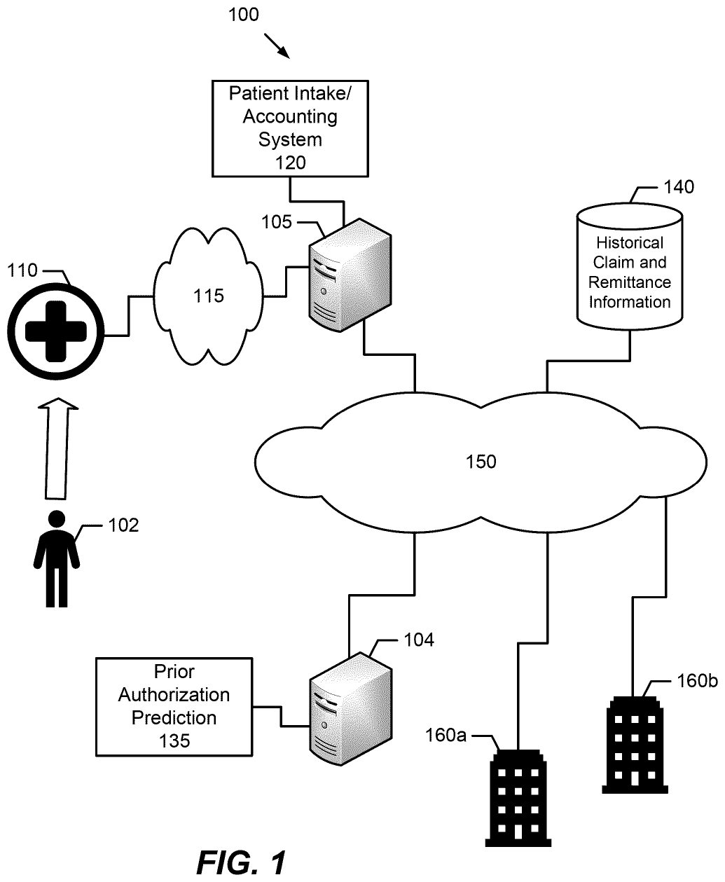

Referring to FIG. 1, a communication network 100 including a prior authorization prediction system, in accordance with some embodiments of the disclosure, comprises one or more health care provider facilities or practices 110. Each health care provider facility or practice 110 may represent various types of organizations that are used to deliver health care services to patients 102 via health care professionals, which are referred to generally herein as “providers.” The providers may include, but are not limited to, hospitals, medical practices, mobile patient care facilities, diagnostic centers, lab centers, pharmacies, and the like. The providers may operate by providing health care services for patients and then invoicing one or more payors 160a and 160b for the services rendered. The payors 160a and 160b may include, but are not limited to, providers of private insurance plans, providers of government insurance plans (e.g., Medicare, Medicaid, state, or federal public employee insurance plans), providers of hybrid insurance plans (e.g., Affordable Care Act plans), private of private medical cost sharing plans, and the patients themselves. The provider facility 110 includes a patient intake/accounting system server 105 accessible via a network 115. The patient intake/accounting system server 105 is configured with a patient intake/accounting system module 120 to manage the intake of patients for appointments and to generate invoices for payors for services and products rendered through the provider 110. The network 115 communicatively couples the patient intake/accounting system server 105 to other devices, terminals, and systems in the provider's facility 110. The network 115 may comprise one or more local or wireless networks to communicate with the patient intake/accounting system server 105 when the patient intake/accounting system server 105 is located in or proximate to the health care service provider facility 110. When the patient intake/accounting system server 105 is in a remote location from the health care facility, such as part of a cloud computing system or at a central computing center, then the network 115 may include one or more wide area or global networks, such as the Internet.

According to embodiments of the disclosure, a prior authorization prediction system server 104 may include a prior authorization prediction module 135 that is configured to process historical claim and claim remittance information for various payors, providers, and procedures, which are collected and stored in the historical claim and remittance information repository 140, perform a statistical analysis on this historical information, and generate a prior authorization model that can be used to predict whether a particular procedure will require prior authorization based on a specific payor's prior authorization rules.

A network 150 couples the provider patient intake/accounting system server 105 and payors 160a, 160b to the prior authorization prediction system server 104. The network 150 may be a global network, such as the Internet or other publicly accessible network. Various elements of the network 150 may be interconnected by a wide area network, a local area network, an Intranet, and/or other private network, which may not be accessible by the general public. Thus, the communication network 150 may represent a combination of public and private networks or a virtual private network (VPN). The network 150 may be a wireless network, a wireline network, or may be a combination of both wireless and wireline networks.

The prior authorization prediction service provided through the prior authorization prediction server 130 and the prior authorization prediction module 135 for predicting when prior authorization is required for a health care procedure using statistical analytics may, in some embodiments, be embodied as a cloud service. For example, health care service providers and/or payors may access the claim enrichment system as a Web service. In some embodiments, the claim enrichment system service may be implemented as a Representational State Transfer Web Service (RESTful Web service).

Although FIG. 1 illustrates an example communication network including a prior authorization prediction service for predicting when prior authorization is required for a health care procedure using statistical analytics, it will be understood that embodiments of the inventive subject matter are not limited to such configurations, but are intended to encompass any configuration capable of carrying out the operations described herein.

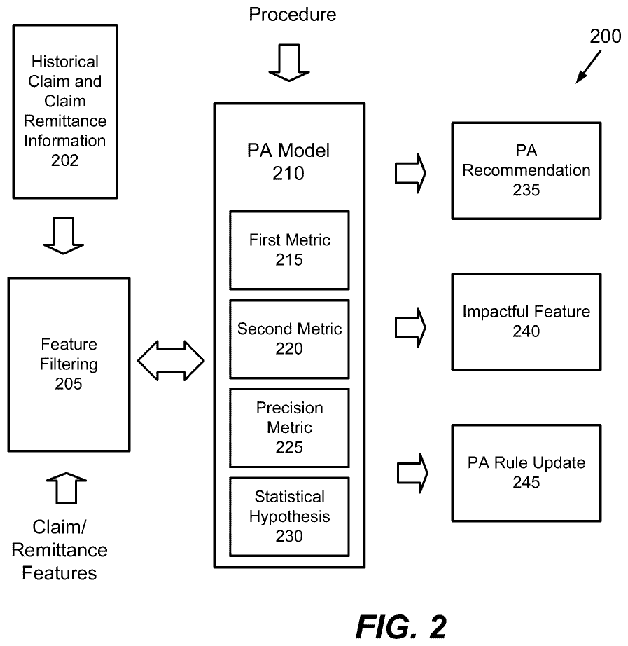

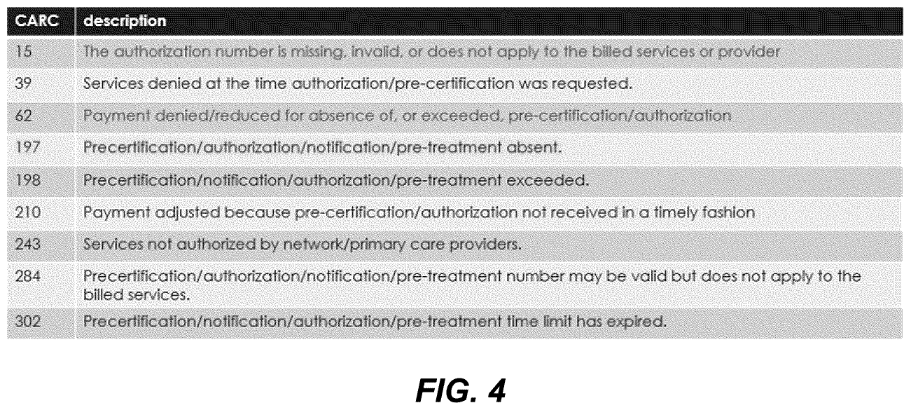

FIG. 2 is a block diagram that illustrates the prior authorization prediction system provided by the prior authorization prediction system server 103 and prior authorization prediction module 135 in accordance with some embodiments of the disclosure. As shown in FIG. 2, the prior authorization prediction system 200 includes a plurality of modules that are coupled in pipeline fashion. The prior authorization prediction system 200 uses historical claim information along with claim remittance information 202 to generate a Prior Authorization (PA) model 210 that can be used to predict whether a particular procedure requires prior authorization based on a rule or policy used by a particular payor. The claim and remittance information 202 may be associated with a variety of different payors and include information on multiple types of procedures performed by providers and remittance information generated by payors for those procedures. The remittance information may include, but is not limited to Claim Adjustment Reason Code (CARC) information, Remittance Advice Remark Code (RARC) information, claim approval information, claim denial information, and the like. FIG. 4 provides a chart with example CARC information that can be included by a payor when processing a claim to assist a provider, for example, in correcting claim errors or deficiencies. RARC information is similar to CARC information, but provides additional detail. As not all of the historical claim and remittance information may be useful in generating the PA model 210, a feature filtering module 205 may select a subset of the historical claim and remittance information 202 that will be used to generate the PA model 210. For example, a subset of the historical claim and claim remittance information may be selected using the feature filtering module 205 that is targeted to a particular payor, one or more providers, specific patient demographics, specific procedures, and the like. Thus, the claim/remittance features used to select the historical claim and claim remittance information 202 used to generate the PA model 210 may include, but are not limited to, procedure code, payor name, payor identification, CARC information, RARC information, principal diagnosis code, other diagnosis code, place of service, patient age, service year, service month, rendering provider taxonomy code, submitter identification, billing provider National Provider Identification (NPI), rendering provider NPI, and/or referring provider NPI.

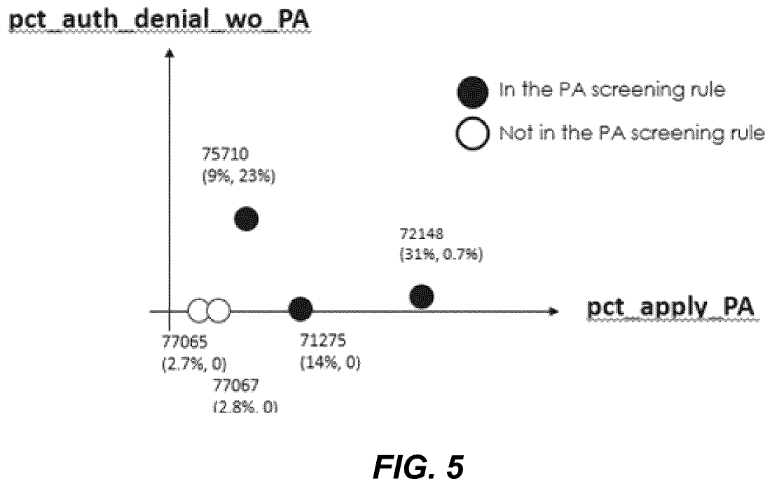

Prior authorization data may, therefore, be extracted from the selected historical claim and claim remittance information 202 output from the feature filtering module 205 and a probabilistic analysis, such as a Bayesian statistical analysis or unsupervised machine learning analysis, may be performed thereon to generate a first metric 215 and a second metric 220 of the PA model for procedure and payor prior authorization rule combinations associated with specific payors. As will be described in further detail below, FIG. 5 is a graph in which the horizontal axis corresponds to the first metric 215 and the vertical axis corresponds to the second metric 220. For three procedures 75710, 71275, and 72148 a prior authorization rule for a payor is deemed as being applicable based on their percentage scores for one or both of the first metric 215 and second metric 220. For two other procedures—77065 and 77067—the prior authorization rule for the payor is deemed as being inapplicable based on their percentage scores for both of the first metric 215 and second metric 220.

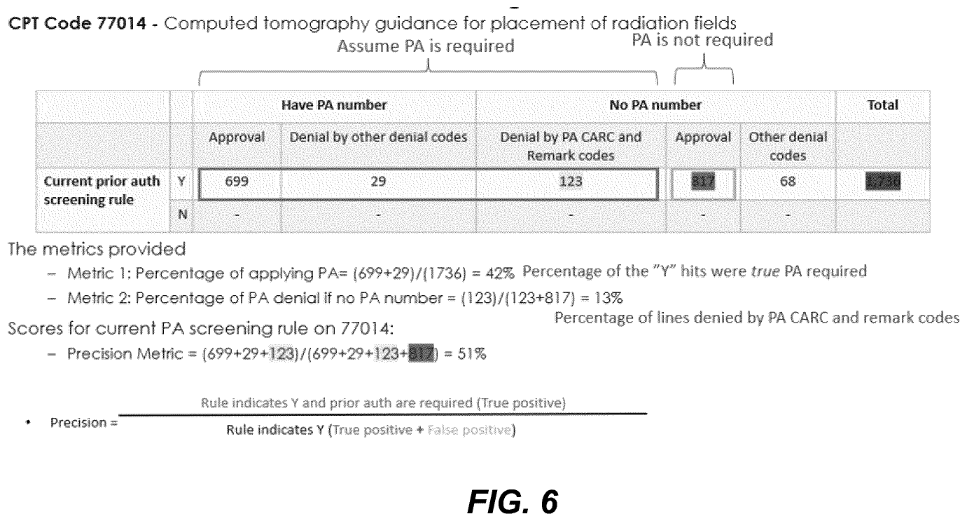

FIG. 6 is a table illustrating calculation of the first and second metrics 215 and 220 along with a precision metric 225 used in generating a prior authorization prediction for a rule in which prior authorization is required in accordance with some embodiments of the disclosure. Referring to FIG. 6, for a procedure corresponding to CPT code 77014, the PA model 210 either receives a rule, electronically, for example, from the payor for this procedure or the PA model 210 derives, based on the first and second metrics 215 and 220, that the payor has a rule that prior authorization is required. For example, the first metric 215 is computed as a percentage of claims that are submitted to the payor for this procedure in which prior authorization was requested. In the example, this number is 42% (699+29/1746). The second metric 220 is computed as a percentage of claims denied when prior authorization has not been requested. In the example, this number is 13% (123/(123+817)). In this example, the two metrics give somewhat conflicting information. Providers seek prior authorization for this procedure a relatively high percentage of the time (42%). Yet the payor infrequently denies payment if prior authorization is not sought as claims without prior authorization for this procedure are only denied 13% of the time. As a result, the PA model 210 may generate a third metric 225 that may be indicative of how consistent a particular prior authorization rule is being enforced by a payor. The precision metric 225 for a rule that is interpreted as requiring prior authorization is given by a quotient where a number of true positive events for the prior authorization rule is the dividend and a sum of the true positive events plus false positive events for the prior authorization rule is the divisor. In the example shown in FIG. 6, the precision metric 225 is computed using the first and second metric 215 and 220 information as 51%, which reflects the inconsistent manner in which the prior authorization rule is enforced by the payor or the lack of understanding of the prior authorization rule by the providers as a significant number of claims are submitted in which prior authorization was obtained, but the payor approves a large percentage of claims in which prior authorization was not obtained. In some embodiments, the precision metric 225 may be evaluated relative to a precision metric threshold. If the precision metric 225 satisfies the threshold, e.g., the precision is less than 75%, then a recommendation may be made to modify the prior authorization rule to clarify its operation so that it is enforced in a more consistent basis and/or providers are provided with clearer information on the prior authorization rule so they better understand whether prior authorization is required. In some embodiments, the payor may allow the prior authorization rule to be automatically modified responsive to generation of the recommendation. For example, the recommendation for modifying the prior authorization rule may be electronically communicated to the payor. A request may be received from the payor to proceed with modifying the prior authorization rule. The prior authorization rule may then be modified in response to the modification request received from the payor.

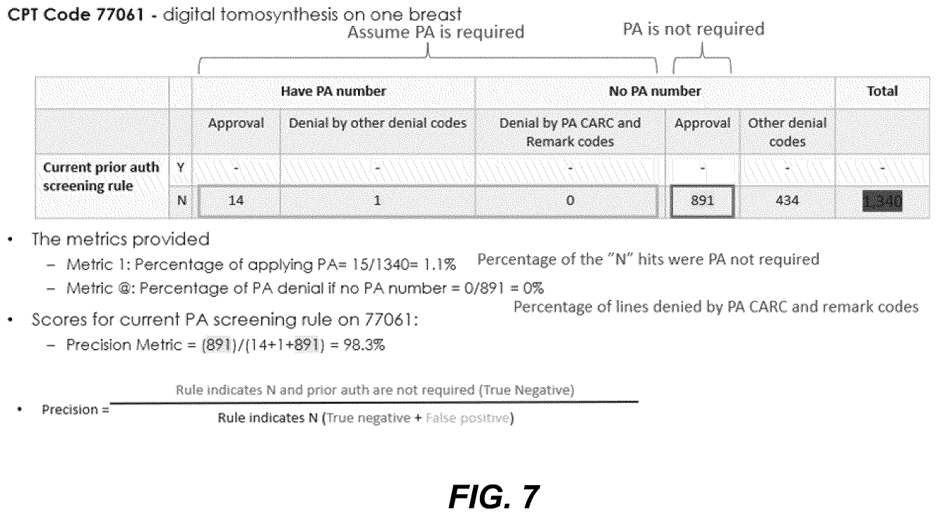

FIG. 7 is a table illustrating calculation of the first and second metrics 215 and 220 along with a precision metric 225 used in generating a prior authorization prediction for a rule in which prior authorization is not required in accordance with some embodiments of the disclosure. Referring to FIG. 7, for a procedure corresponding to CPT code 77061, the PA model 210 either receives a rule, electronically, for example, from the payor for this procedure or the PA model 210 derives, based on the first and second metrics 215 and 220, that the payor has a rule that prior authorization is not required. For example, the first metric 215 is computed as a percentage of claims that are submitted to the payor for this procedure in which prior authorization was requested. In the example, this number is 1.1% (14+1/1340). The second metric 220 is computed as a percentage of claims denied when prior authorization has not been requested. In the example, this number is 0% (0/891). In this example, the two metrics give consistent information. Providers seek prior authorization for this procedure a very low percentage of the time (1.1%). Moreover, the payor has never denied payment if prior authorization is not sought. The precision metric 225 for a rule that is interpreted as not requiring prior authorization is given by a quotient where a number of true negative events for the prior authorization rule is the dividend and a sum of the true negative events plus false positive events for the prior authorization rule is the divisor. In the example shown in FIG. 7, the precision metric 225 is computed using the first and second metric 215 and 220 information as 98.3%, which reflects the consistent manner in which the prior authorization rule is enforced by the payor and the generally clear understanding of the prior authorization rule by the providers as the providers rarely seek prior authorization for this procedure. Due to the high precision metric percentage value relative to a precision metric threshold, the precision metric 225 would be unlikely to trigger a recommendation to modify the prior authorization rule as the prior authorization rule is consistently enforced and the a prediction that no prior authorization is required by this payor for this procedure can be predicted with high confidence based on the first metric 215 and the second metric 220.

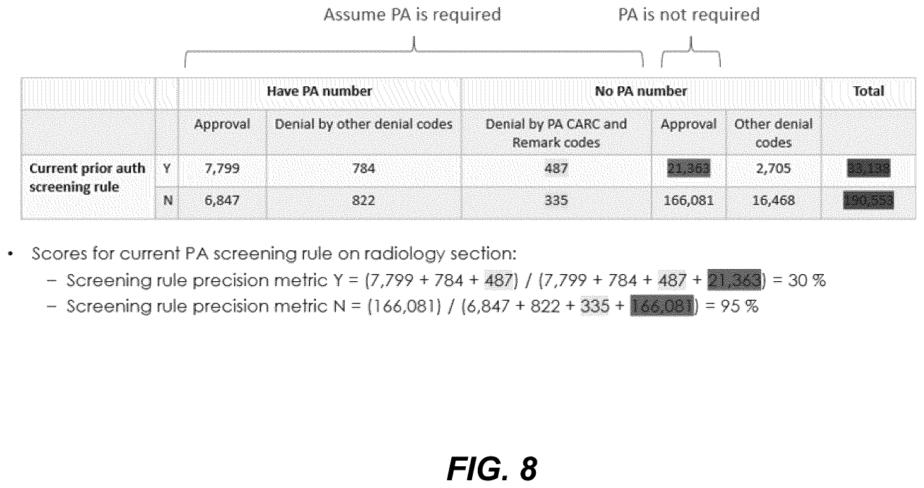

In some embodiments of the disclosure, the first and second metric 215 and 220 statistics may be accumulated across multiple prior authorization defined by a payor and a precision metric 225 may be computed for a compilation of rules. FIG. 8 is a table illustrating calculation of precision metrics 225 for multiple prior authorization rules associated with a particular health care area in accordance with some embodiments of the disclosure. In the example of FIG. 8, first and second metric 215 and 220 statistics are accumulate for multiple prior authorization rules in the radiology area including rules for procedures in which prior authorization is required and rules in which prior authorization is not required. As shown in the example of FIG. 8, the rules for procedures in which prior authorization is required has a relatively low precision metric of 30% while rules for procedures in which prior authorization is not required has a relatively high precision metric of 95%. Thus, a recommendation may be generated for the provider to review and/or modify one or more prior authorization rules in the radiology area in which prior authorization is presumed to be required to allow for more consistent enforcement and/or to better communicate to providers that prior authorization is not required in many circumstances due to the large number of approvals without prior authorization despite the assumption that prior authorization is required.

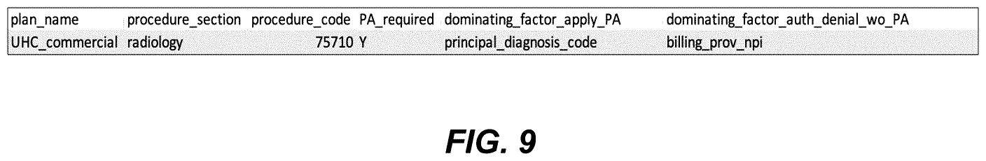

Returning to FIG. 2, a statistical a statistical hypothesis module 230 may be configured to determine which features from among the claim/remittance features used in filtering the historical claim and claim remittance information 202 are the most impactful in the generating the first metric 215 and second metric 220 results. For example, in some embodiments, a chi-squared test can be performed to identify a feature from among a plurality of features including, but not limited to, procedure code, payor name, payor identification, CARC information, RARC information, principal diagnosis code, other diagnosis code, place of service, patient age, service year, service month, rendering provider taxonomy code, submitter identification, billing provider National Provider Identification (NPI), rendering provider NPI, and/or referring provider NPI, that is most impactful in contributing to each of the first metric 215 determination and second metric 220 determination. This is illustrated, for example, in FIG. 9, which shows the principal diagnosis code as being the most impactful feature in contributing to the first metric 215 and the billing provider NPI as being the most impactful feature in contributing to the second metric 220 for a particular procedure and payor plan.

Thus, the PA model 210 may be configured to receive, electronically, for example, information on a procedure from, for example, a provider or other party, and determine the first metric 215, second metric 220, precision metric 225, and perform a statistical hypothesis analysis based on various features to generate a prior authorization recommendation 235, an impactful feature identification 240 for each of the first and second metrics 215 and 220, and, in some circumstances, a prior authorization rule update recommendation 245. The first metric 215 may be evaluated to determine whether it satisfies a first prior authorization threshold and the second metric 220 may be evaluated whether it satisfies a second prior authorization threshold. Two satisfaction scores may be determined for the first and second metrics 215 and 220, respectively, and the combined score may be used to determine whether prior authorization is recommended for this procedure and this payor and insurance plan. In other embodiments, the two metrics 215 and 220 may be combined in advance using for example, an average, weighted average, or other mathematical combination. The combined score may be evaluated to determine whether the combined score satisfies a prior authorization threshold. Based on this evaluation, a prior authorization recommendation may be made for this procedure and this payor and insurance plan. In some embodiments, the recommendation may include a recommendation to delay performing the procedure until the procedure is authorized by the payor to reduce the risk of a payor declining to cover a performed procedure. The precision metric 225 may also be provided, which may provide a measure of confidence with respect to the generated prior authorization recommendation, which is based on a prediction of whether prior authorization is required. The most impactful features 240 may be identified for each of the first and second metrics 215 and 220, which may be used by a provider, payor, or other party to give heightened scrutiny for procedures or claims in which these features are implicated as they have the greatest potential to impact whether prior authorization is required. As described above, a recommendation may be made to update a prior authorization rule 245 when the precision metric for the rule satisfies a precision metric threshold. Advantageously, may increase consistency in enforcement of a particular rule and/or improve the clarity of a rule so that providers do not spend unnecessary manual and computing resources in obtaining prior authorizations for procedures that are not required.

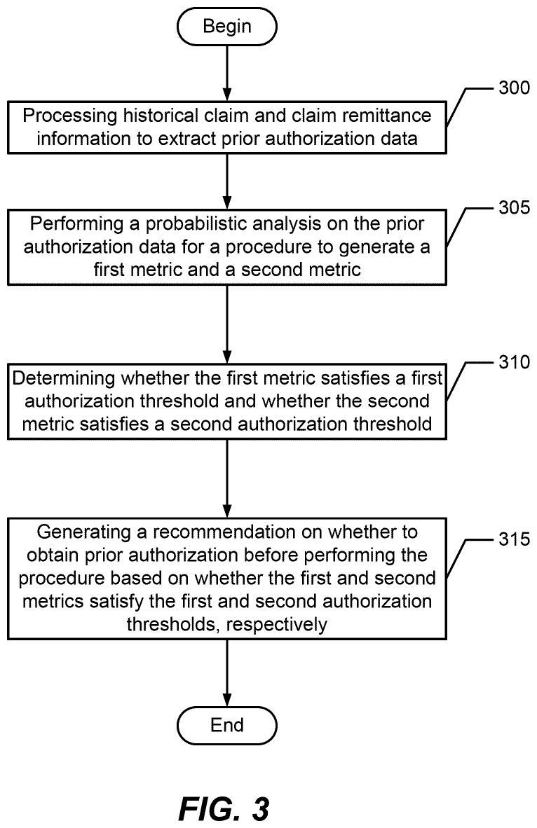

FIG. 3 is a flowchart that illustrates operations prior authorization prediction system in accordance with some embodiments of the disclosure. Referring now to FIG. 3, operations begin at block 300 where historical claim and claim remittance information 202 is processed to extra prior authorization data. A probabilistic analysis is performed at block 305 on the prior authorization data to generate a first metric and a second metric 215 and 220. In accordance with various embodiments of the disclosure, the probabilistic analysis may be based on conditional probabilities. In some embodiments, the probabilistic analysis may be a Bayesian statistical analysis. In other embodiments, the probabilistic analysis may be an unsupervised machine learning analysis. A determination is made whether the first metric 215 satisfies a first authorization threshold and whether the second metric 220 satisfies a second authorization threshold at block 310. A recommendation is generated at block 315 on whether to obtain prior authorization before performing the procedure based on whether the first and second metrics satisfy the first and second authorization thresholds, respectively.

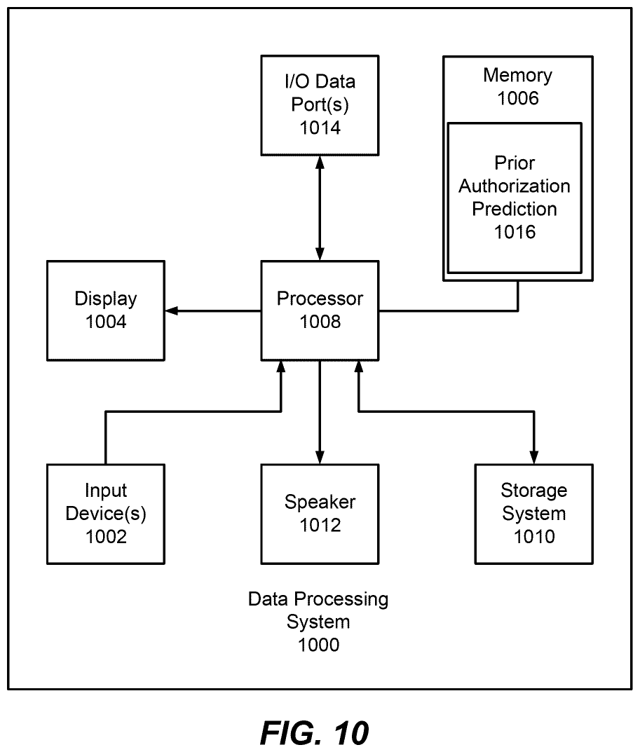

Referring now to FIG. 10, a data processing system 1000 that may be used to implement the prior authorization prediction system server 104 of FIG. 1, in accordance with some embodiments of the disclosure, comprises input device(s) 1002, such as a keyboard or keypad, a display 1004, and a memory 1006 that communicate with a processor 1008. The data processing system 1000 may further include a storage system 1010, a speaker 1012, and an input/output (I/O) data port(s) 1014 that also communicate with the processor 1008. The storage system 1010 may include removable and/or fixed media, such as floppy disks, ZIP drives, hard disks, or the like, as well as virtual storage, such as a RAMDISK. The I/O data port(s) 1014 may be used to transfer information between the data processing system 1000 and another computer system or a network (e.g., the Internet). The memory 1006 may be configured with a prior authorization prediction module 1016 that may provide functionality that may include, but is not limited to, predicting when prior authorization is required for a health care procedure using statistical analytics. The components of the data processing system 1000 when configured with the prior authorization prediction module 1016 may provide a special purpose computing system that is configured to predict when a health care procedure requires prior authorization before it is performed. As a result, the data processing system 1000 and prior authorization prediction system server 104 may be used to control the timing of the performance of a health care procedure.

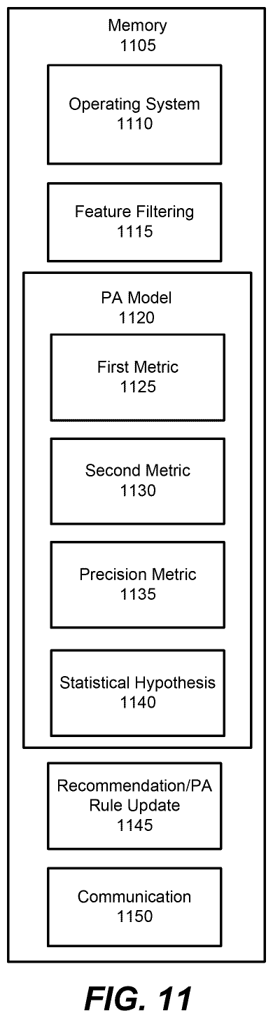

FIG. 11 illustrates a memory 1105 that may be used in embodiments of data processing systems, such as the prior authorization prediction server 104 of FIG. 1 and the data processing system of FIG. 10, respectively, to facilitate predicting when prior authorization is required for a health care procedure using statistical analytics. The memory 1105 is representative of the one or more memory devices containing the software and data used for facilitating operations of the prior authorization prediction server 104 and the prior authorization prediction module 135 as described herein. The memory 1105 may include, but is not limited to, the following types of devices: cache, ROM, PROM, EPROM, EEPROM, flash, SRAM, and DRAM. As shown in FIG. 11, the memory 1105 may contain five or more categories of software and/or data: an operating system 1110, a feature filtering module 1115, a PA model 1120, a recommendation/PA rule update module 1145, and a communication module 1150. In particular, the operating system 1110 may manage the data processing system's software and/or hardware resources and may coordinate execution of programs by the processor.

The feature filtering module 1115 may be configured may be configured to perform one or more of the operations described above with respect to the feature filtering module 205 of FIG. 2 and FIGS. 3-9. The PA model 1120 includes a first metric module 1125, a second metric module 1130, a precision metric module 1135, and a statistical hypothesis module 1140. The first metric module 1125 may be configured to perform one or more of the operations described above with respect to the first metric module 215 of FIG. 2 and FIGS. 3-9. The second metric module 1130 may be configured to perform one or more of the operations described above with respect to the second metric module 220 of FIG. 2 and FIGS. 3-9. The precision metric module 1135 may be configured to perform one or more of the operations described above with respect to the precision metric module 225 of FIG. 2 and FIGS. 3-9. The statistical hypothesis module 1140 may be configured to perform one or more of the operations described above with respect to the statistical hypothesis module 230 of FIG. 2 and FIGS. 3-9. The recommendation/PA rule update module 1145 may be configured to perform one or more of the operations described above with respect to the PA rule update block 245 of FIG. 2 and FIGS. 3-9. The communication module 1150 may be configured to facilitate communication between the prior authorization prediction system server 104, the provider 110, and payors 160a, 160b of FIG. 1.

Although FIGS. 10 and 11 illustrate hardware/software architectures that may be used in data processing systems, such as the prior authorization prediction server 104 of FIG. 1, in accordance with some embodiments of the disclosure, it will be understood that the present invention is not limited to such a configuration but is intended to encompass any configuration capable of carrying out operations described herein.

Computer program code for carrying out operations of data processing systems discussed above with respect to FIGS. 1-10 may be written in a high-level programming language, such as Python, Java, C, and/or C++, for development convenience. In addition, computer program code for carrying out operations of the present invention may also be written in other programming languages, such as, but not limited to, interpreted languages. Some modules or routines may be written in assembly language or even micro-code to enhance performance and/or memory usage. It will be further appreciated that the functionality of any or all of the program modules may also be implemented using discrete hardware components, one or more application specific integrated circuits (ASICs), or a programmed digital signal processor or microcontroller.

Moreover, the functionality of the prior authorization prediction system server 104 of FIG. 1 and the data processing system of FIG. 10 may each be implemented as a single processor system, a multi-processor system, a multi-core processor system, or even a network of stand-alone computer systems, in accordance with various embodiments of the disclosure. Each of these processor/computer systems may be referred to as a “processor” or “data processing system.”

The data processing apparatus described herein with respect to FIGS. 1-11 may be used to facilitate predicting when prior authorization is required for a health care procedure using statistical analytics according to some embodiments of the disclosure described herein. These apparatus may be embodied as one or more enterprise, application, personal, pervasive and/or embedded computer systems and/or apparatus that are operable to receive, transmit, process and store data using any suitable combination of software, firmware and/or hardware and that may be standalone or interconnected by any public and/or private, real and/or virtual, wired and/or wireless network including all or a portion of the global communication network known as the Internet, and may include various types of tangible, non-transitory computer readable media. In particular, the memory 1105 when coupled to a processor includes computer readable program code that, when executed by the processor, causes the processor to perform operations including one or more of the operations described herein with respect to FIGS. 1-9.

Some embodiments of the disclosure may provide a prior authorization prediction system that incorporates statistical analytics and hypothesis testing that advantageously can improve efficiency and reduce costs for providers when determining whether prior authorization is required for a procedure by eliminating manual tasks performed to obtain prior authorization information from payors. Advantageously, this may improve accuracy in generating claims thereby reducing processing loads on both provider claim generation systems and payor claim processing systems.

Some embodiments of the disclosure may provide a decision support system for detecting fraud, such as predicting whether a dental claim is fraudulent as set forth by the following examples: Example 1: a computer-implemented method comprises: processing, by one or more processors, historical claim and claim remittance information to extract prior authorization data; performing, by the one or more processors, a Bayesian statistical analysis on the prior authorization data for a procedure to generate a first metric and a second metric, the first metric comprising a percentage of claims including the procedure in which prior authorization is applied for and the second metric comprising a percentage of claims including the procedure in which prior authorization was not applied for and were denied payment for not applying for prior authorization; determining, by the one or more processors, whether the first metric satisfies a first authorization threshold and the second metric satisfies a second authorization threshold, the first and second thresholds being associated with a prior authorization rule corresponding to the procedure; and generating, by the one or more processors, a recommendation on whether to obtain prior authorization before performing the procedure based on whether the first metric satisfies the first authorization threshold and the second metric satisfies the second authorization threshold.

Example 2: the computer-implemented method of Example 1, wherein the method further comprises: electronically receiving, by the one or more processors, a prior authorization inquiry from a provider regarding the procedure; and electronically communicating, by the one or more processors, the recommendation to the provider in response to receiving the inquiry.

Example 3: the computer-implemented method of any of Examples 1 and 2, wherein the recommendation comprises a recommendation to delay performing the procedure until the procedure is authorized by a payor.

Example 4: the computer-implemented method of any of Examples 1-3, wherein the method further comprises: generating, by the one or more processors, a precision metric for the prior authorization rule; wherein the precision metric is given by a first quotient where a number of true positive events for the prior authorization rule is the dividend and a sum of true positive events plus false positive events for the prior authorization rule is the divisor when the prior authorization rule specifies that prior authorization is required; and wherein the precision metric is given by a second quotient where a number of true negative events for the prior authorization rule is the dividend and a sum of true negative events plus false positive events for the prior authorization rule is the divisor when the prior authorization rule specifies that prior authorization is not required.

Example 5: the computer-implemented method of any of Examples 1-4, wherein the method further comprises: determining, by the one or more processors, the prior authorization rule based on the first metric and the second metric.

Example 6: the computer-implemented method of any of Examples 1-5, wherein the method further comprises: electronically receiving, by the one or more processors, the prior authorization rule from a payor.

Example 7: the computer-implemented method of any of Examples 4-6, wherein the method further comprises: determining, by the one or more processors, whether the precision metric satisfies a precision metric threshold; and generating, by the one or more processors, a recommendation for modifying the prior authorization rule when the precision metric satisfies the precision metric threshold.

Example 8: the computer-implemented method of Example 7, wherein the method further comprises: electronically modifying, by the one or more processors, the prior authorization rule responsive to generating the recommendation for modifying the prior authorization rule.

Example 9: the computer-implemented method of any of Examples 7 and 8, wherein the method further comprises: electronically communicating, by the one or more processors, the recommendation for modifying the prior authorization rule to a payor; electronically receiving, by the one or more processors, a request from the payor to modify the prior authorization rule; and electronically modifying, by the one or more processors, the prior authorization rule responsive to receiving the request from the payor to modify the prior authorization rule.

Example 10: the computer-implemented method of any of Examples 1-9, wherein the method further comprises: filtering, by the one or more processors, the historical claim and claim remittance information based on a plurality of features to remove a portion of the historical claim and claim remittance information leaving a subset of the historical claim and claim remittance information; wherein processing the historical claim and claim remittance information to extract the prior authorization data comprises: processing, by the one or more processors, the subset of the historical claim and claim remittance information to extract the prior authorization data.

Example 11: the computer-implemented method of Example 10, wherein the plurality of features comprise procedure code, payor name, payor identification, claim adjustment reason code, remittance advice remark code, principal diagnosis code, other diagnosis code, place of service, patient age, service year, service month, rendering provider taxonomy code, submitter identification, billing provider National Provider Identification (NPI), rendering provider NPI, or referring provider NPI.

Example 12: the computer-implemented method of any of Examples 10 and 11, wherein the method further comprises: performing, by the one or more processors, a first statistical hypothesis test on the plurality of features to identify a first one of the plurality of features having a greatest impact on the first metric; and performing, by the one or more processors, a second statistical hypothesis test on the plurality of features to identify a second one of the plurality of features having a greatest impact on the second metric.

Example 13: the computer-implemented method of Example 12, wherein the statistical hypothesis test is a chi-squared test.

Example 14: the computer-implemented method of any of Examples 1-13, wherein the probabilistic analysis is a Bayesian statistical analysis or an unsupervised machine learning analysis.

Example 15: a system comprises: one or more processors; and a memory coupled to the one or more processors and comprising computer readable program code embodied in the memory that is executable by the processor to perform operations comprising: processing, by the one or more processors, historical claim and claim remittance information to extract prior authorization data; performing, by the one or more processors, a Bayesian statistical analysis on the prior authorization data for a procedure to generate a first metric and a second metric, the first metric comprising a percentage of claims including the procedure in which prior authorization is applied for and the second metric comprising a percentage of claims including the procedure in which prior authorization was not applied for and were denied payment for not applying for prior authorization; determining, by the one or more processors, whether the first metric satisfies a first authorization threshold and the second metric satisfies a second authorization threshold, the first and second thresholds being associated with a prior authorization rule corresponding to the procedure; and generating, by the one or more processors, a recommendation on whether to obtain prior authorization before performing the procedure based on whether the first metric satisfies the first authorization threshold and the second metric satisfies the second authorization threshold.

Example 16: the system of Example 15, wherein the operations further comprise: generating, by the one or more processors, a precision metric for the prior authorization rule; wherein the precision metric is given by a first quotient where a number of true positive events for the prior authorization rule is the dividend and a sum of true positive events plus false positive events for the prior authorization rule is the divisor when the prior authorization rule specifies that prior authorization is required; and wherein the precision metric is given by a second quotient where a number of true negative events for the prior authorization rule is the dividend and a sum of true negative events plus false positive events for the prior authorization rule is the divisor when the prior authorization rule specifics that prior authorization is not required.

Example 17: the system of Example 16, wherein the operations further comprise: determining, by the one or more processors, whether the precision metric satisfies a precision metric threshold; and generating, by the one or more processors, a recommendation for modifying the prior authorization rule when the precision metric satisfies the precision metric threshold.

Example 18: the system of any of Examples 15-17, wherein the operations further comprise: filtering, by the one or more processors, the historical claim and claim remittance information based on a plurality of features to remove a portion of the historical claim and claim remittance information leaving a subset of the historical claim and claim remittance information; wherein processing the historical claim and claim remittance information to extract the prior authorization data comprises: processing, by the one or more processors, the subset of the historical claim and claim remittance information to extract the prior authorization data.

Example 19: the system of Example 18, wherein the plurality of features comprise procedure code, payor name, payor identification, claim adjustment reason code, remittance advice remark code, principal diagnosis code, other diagnosis code, place of service, patient age, service year, service month, rendering provider taxonomy code, submitter identification, billing provider National Provider Identification (NPI), rendering provider NPI, or referring provider NPI; and wherein the operations further comprise: performing, by the one or more processors, a first statistical hypothesis test on the plurality of features to identify a first one of the plurality of features having a greatest impact on the first metric; and performing, by the one or more processors, a second statistical hypothesis test on the plurality of features to identify a second one of the plurality of features having a greatest impact on the second metric.

Example 20: a computer program product comprises one or more non-transitory computer readable storage media including instructions that, when executed by one or more processors, cause the one or more processors to: process, by the one or more processors, historical claim and claim remittance information to extract prior authorization data; perform, by the one or more processors, a Bayesian statistical analysis on the prior authorization data for a procedure to generate a first metric and a second metric, the first metric comprising a percentage of claims including the procedure in which prior authorization is applied for and the second metric comprising a percentage of claims including the procedure in which prior authorization was not applied for and were denied payment for not applying for prior authorization; determine, by the one or more processors, whether the first metric satisfies a first authorization threshold and the second metric satisfies a second authorization threshold, the first and second thresholds being associated with a prior authorization rule corresponding to the procedure; and generate, by the one or more processors, a recommendation on whether to obtain prior authorization before performing the procedure based on whether the first metric satisfies the first authorization threshold and the second metric satisfies the second authorization threshold.

### FURTHER DEFINITIONS AND EMBODIMENTS

In the above-description of various embodiments of the present disclosure, it is to be understood that the terminology used herein is for the purpose of describing particular embodiments only and is not intended to be limiting of the invention. Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of this specification and the relevant art and will not be interpreted in an idealized or overly formal sense expressly so defined herein.

The flowchart and block diagrams in the figures illustrate the architecture, functionality, and operation of possible implementations of systems, methods, and computer program products according to various aspects of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular aspects only and is not intended to be limiting of the inventive concept. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Like reference numbers signify like elements throughout the description of the figures.

In the above-description of various embodiments of the present disclosure, aspects of the present disclosure may be illustrated and described herein in any of a number of patentable classes or contexts including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present disclosure may take the form of a computer program product comprising one or more computer readable media having computer readable program code embodied thereon.

Any combination of one or more computer readable media may be used. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

The description of the present inventive concept has been presented for purposes of illustration and description, but is not intended to be exhaustive or limited to the disclosure in the form disclosed. Many modifications and variations will be apparent to those of ordinary skill in the art without departing from the scope and spirit of the inventive concept. The aspects of the disclosure herein were chosen and described to best explain the principles of the inventive concept and the practical application, and to enable others of ordinary skill in the art to understand the inventive concept with various modifications as are suited to the particular use contemplated.

What is claimed is:

1. A computer-implemented method, comprising:
processing, by one or more processors, historical claim and claim remittance information to extract prior authorization data;
performing, by the one or more processors, a probabilistic analysis on the prior authorization data for a procedure to generate a first metric and a second metric, the first metric comprising a percentage of claims including the procedure in which prior authorization is applied for and the second metric comprising a percentage of claims including the procedure in which prior authorization was not applied for and were denied payment for not applying for prior authorization;
determining, by the one or more processors, whether the first metric satisfies a first authorization threshold and the second metric satisfies a second authorization threshold, the first and second thresholds being associated with a prior authorization rule corresponding to the procedure; and
generating, by the one or more processors, a recommendation on whether to obtain prior authorization before performing the procedure based on whether the first metric satisfies the first authorization threshold and the second metric satisfies the second authorization threshold.

2. The computer-implemented method of claim 1, further comprising:
electronically receiving, by the one or more processors, a prior authorization inquiry from a provider regarding the procedure; and
electronically communicating, by the one or more processors, the recommendation to the provider in response to receiving the inquiry.

3. The computer-implemented method of claim 2, wherein the recommendation comprises a recommendation to delay performing the procedure until the procedure is authorized by a payor.

4. The computer-implemented method of claim 1, further comprising:
generating, by the one or more processors, a precision metric for the prior authorization rule;
wherein the precision metric is given by a first quotient where a number of true positive events for the prior authorization rule is the dividend and a sum of true positive events plus false positive events for the prior authorization rule is the divisor when the prior authorization rule specifies that prior authorization is required; and
wherein the precision metric is given by a second quotient where a number of true negative events for the prior authorization rule is the dividend and a sum of true negative events plus false positive events for the prior authorization rule is the divisor when the prior authorization rule specifies that prior authorization is not required.

5. The computer-implemented method of claim 4, further comprising:
determining, by the one or more processors, the prior authorization rule based on the first metric and the second metric.

6. The computer-implemented method of claim 4, further comprising:
electronically receiving, by the one or more processors, the prior authorization rule from a payor.

7. The computer-implemented method of claim 4, further comprising:
determining, by the one or more processors, whether the precision metric satisfies a precision metric threshold; and
generating, by the one or more processors, a recommendation for modifying the prior authorization rule when the precision metric satisfies the precision metric threshold.

8. The computer-implemented method of claim 7, further comprising:
electronically modifying, by the one or more processors, the prior authorization rule responsive to generating the recommendation for modifying the prior authorization rule.

9. The computer-implemented method of claim 7, further comprising:
electronically communicating, by the one or more processors, the recommendation for modifying the prior authorization rule to a payor;
electronically receiving, by the one or more processors, a request from the payor to modify the prior authorization rule; and
electronically modifying, by the one or more processors, the prior authorization rule responsive to receiving the request from the payor to modify the prior authorization rule.

10. The computer-implemented method of claim 1, further comprising:
filtering, by the one or more processors, the historical claim and claim remittance information based on a plurality of features to remove a portion of the historical claim and claim remittance information leaving a subset of the historical claim and claim remittance information;
wherein processing the historical claim and claim remittance information to extract the prior authorization data comprises:
processing, by the one or more processors, the subset of the historical claim and claim remittance information to extract the prior authorization data.

11. The computer-implemented method of claim 10, wherein the plurality of features comprise procedure code, payor name, payor identification, claim adjustment reason code, remittance advice remark code, principal diagnosis code, other diagnosis code, place of service, patient age, service year, service month, rendering provider taxonomy code, submitter identification, billing provider National Provider Identification (NPI), rendering provider NPI, or referring provider NPI.

12. The computer-implemented method of claim 11, further comprising:
performing, by the one or more processors, a first statistical hypothesis test on the plurality of features to identify a first one of the plurality of features having a greatest impact on the first metric; and
performing, by the one or more processors, a second statistical hypothesis test on the plurality of features to identify a second one of the plurality of features having a greatest impact on the second metric.

13. The computer-implemented method of claim 12, wherein the statistical hypothesis test is a chi-squared test.

14. The computer-implemented method of claim 1, wherein the probabilistic analysis is a Bayesian statistical analysis or an unsupervised machine learning analysis.

15. A system, comprising:
one or more processors; and
a memory coupled to the one or more processors and comprising computer readable program code embodied in the memory that is executable by the processor to perform operations comprising:
processing, by the one or more processors, historical claim and claim remittance information to extract prior authorization data;
performing, by the one or more processors, a probabilistic analysis on the prior authorization data for a procedure to generate a first metric and a second metric, the first metric comprising a percentage of claims including the procedure in which prior authorization is applied for and the second metric comprising a percentage of claims including the procedure in which prior authorization was not applied for and were denied payment for not applying for prior authorization;
determining, by the one or more processors, whether the first metric satisfies a first authorization threshold and the second metric satisfies a second authorization threshold, the first and second thresholds being associated with a prior authorization rule corresponding to the procedure; and
generating, by the one or more processors, a recommendation on whether to obtain prior authorization before performing the procedure based on whether the first metric satisfies the first authorization threshold and the second metric satisfies the second authorization threshold.

16. The system of claim 15, wherein the operations further comprise:
generating, by the one or more processors, a precision metric for the prior authorization rule;
wherein the precision metric is given by a first quotient where a number of true positive events for the prior authorization rule is the dividend and a sum of true positive events plus false positive events for the prior authorization rule is the divisor when the prior authorization rule specifies that prior authorization is required; and
wherein the precision metric is given by a second quotient where a number of true negative events for the prior authorization rule is the dividend and a sum of true negative events plus false positive events for the prior authorization rule is the divisor when the prior authorization rule specifies that prior authorization is not required.

17. The system of claim 16, wherein the operations further comprise:
determining, by the one or more processors, whether the precision metric satisfies a precision metric threshold; and
generating, by the one or more processors, a recommendation for modifying the prior authorization rule when the precision metric satisfies the precision metric threshold.

18. The system of claim 15, wherein the operations further comprise:
filtering, by the one or more processors, the historical claim and claim remittance information based on a plurality of features to remove a portion of the historical claim and claim remittance information leaving a subset of the historical claim and claim remittance information;
wherein processing the historical claim and claim remittance information to extract the prior authorization data comprises:
processing, by the one or more processors, the subset of the historical claim and claim remittance information to extract the prior authorization data.

19. The system of claim 18, wherein the plurality of features comprise procedure code, payor name, payor identification, claim adjustment reason code, remittance advice remark code, principal diagnosis code, other diagnosis code, place of service, patient age, service year, service month, rendering provider taxonomy code, submitter identification, billing provider National Provider Identification (NPI), rendering provider NPI, or referring provider NPI; and
wherein the operations further comprise:
performing, by the one or more processors, a first statistical hypothesis test on the plurality of features to identify a first one of the plurality of features having a greatest impact on the first metric; and
performing, by the one or more processors, a second statistical hypothesis test on the plurality of features to identify a second one of the plurality of features having a greatest impact on the second metric.

20. A computer program product, comprising:
one or more non-transitory computer readable storage media including instructions that, when executed by one or more processors, cause the one or more processors to:
process, by the one or more processors, historical claim and claim remittance information to extract prior authorization data;
perform, by the one or more processors, a probabilistic analysis on the prior authorization data for a procedure to generate a first metric and a second metric, the first metric comprising a percentage of claims including the procedure in which prior authorization is applied for and the second metric comprising a percentage of claims including the procedure in which prior authorization was not applied for and were denied payment for not applying for prior authorization;
determine, by the one or more processors, whether the first metric satisfies a first authorization threshold and the second metric satisfies a second authorization threshold, the first and second thresholds being associated with a prior authorization rule corresponding to the procedure; and
generate, by the one or more processors, a recommendation on whether to obtain prior authorization before performing the procedure based on whether the first metric satisfies the first authorization threshold and the second metric satisfies the second authorization threshold.
